(19) United States
(12) Patent Application Publication
(10) Pub. No.: US 2024/0010985 A1
(43) Pub. Date: Jan. 11, 2024

(54) DISSOLVABLE GELATIN-BASED MICROCARRIERS GENERATED THROUGH DROPLET MICROFLUIDICS FOR EXPANSION AND CULTURE OF MESENCHYMAL STROMAL CELL

(71) Applicant: Massachusetts Institute of Technology, Cambridge, MA (US)

(72) Inventors: Krystyn J. Van Vliet, Lexington, MA (US); Ee Xien Ng, Singapore (SG)

(21) Appl. No.: 18/020,419

(22) PCT Filed: Apr. 13, 2021
(86) PCT No.: PCT/US2021/027062

§ 371 Date: Feb. 8, 2023

Related U.S. Application Data
(60) Provisional application No. 63/008,972, filed on Apr. 13, 2020.

(51) Int. Cl. C12N 5/0775; C12N 5/00

(52) U.S. Cl. CPC C12N 5/0663; C12N 5/0075; C12N 2537/10; C12N 2533/54; C12N 2509/00

(57) ABSTRACT

The invention relates to a dissolvable gelatin-based microcarrier generated through droplet microfluidics. Also disclosed herein is a method of manufacturing said microcarrier and its use in the processes of cell culture and cell expansion of cells, such as mesenchymal stromal cells (MSCs).

### FIELD OF INVENTION

This current invention relates to the use of dissolvable Gelatin-based microcarriers for expansion and culture of mesenchymal stromal cells. The invention also relates to method of making said material and its uses.

### BACKGROUND

The listing or discussion of a prior-published document in this specification should not necessarily be taken as an acknowledgement that the document is part of the state of the art or is common general knowledge.

Mesenchymal stromal cells (MSCs) consist of multipotent cells that have the capability to undergo trilineage differentiation into adipo-, chondro- and osteo-mesenchymal tissue lineage. In addition, MSCs have shown promising results in regenerative medicine as they can be readily harvested from various adult tissues and further expanded to treat different diseases. Besides having the capability to act as self-renewing cells to replace damaged tissues, MSCs can also act as a “drug factory” when introduced into the body where the MSCs secrete beneficial factors for tissue regeneration in vivo. Therefore, there is a need to expand MSCs in great quantity, either to produce sources of qualified cells to treat multiple patients (alloegeneic MSC therapy) or to meet the required cell dose per patient depending on the type of diseases.

Conventional 2D planar culture is the current gold standard for expansion of anchorage-dependent cell type such as MSCs, but the associated large physical and fluid volumes required of a planar culture surface are incompatible with cost-efficient cell population scale-up, and can also result in gradients of nutrients and gases which affect cell phenotype (Jossen, V. et al., Mass production of mesenchymal stem stem cells—impact of bioreactor design and flow conditions on proliferation and differentiation. In Cells and Biomaterials in Regenerative Medicine, 2014). Therefore, microcarrier (MC) technology was developed for use in bioreactor-based cell manufacturing to provide attachment surfaces for anchorage-dependent cells to adhere onto and proliferate inside bioreactor vessels. The advantages of MC culture include high surface area to volume ratio which allows more cells to attach per culture volume, scalability for parallel processing, achievable homogeneous environmental condition in a stirred MC suspension culture, and minimal shear stress on cells as MCs are freely moving in a stirred culture (Chen, A. K.-L. et al., Biotechnol. Adv. 2013, 31, 1032-1046). MCs typically take the form of a sphere which has maximum high surface area to volume ratio, and can be macroporous, microporous or nonporous.

The MC technology has been widely used for MSC culture and expansion for cell-based therapy. However, conventional MCs focus on optimizing attachment efficiency and proliferation rate of cells on the MC particles yet in the field of cell manufacturing, the limitation lies in the harvesting and separation of cells from the MC particles where yield of viable cells of known therapeutic value can be compromised in those steps. Moreover, the application of conventional MCs for MSC manufacturing is associated with an additional cell-particle separation step that is time-consuming and poses a risk of contamination with the incorporation of another microfluidic module into the cell manufacturing workflow. Hence, the bottleneck of conventional MC technology lies in the cell recovery efficiency during the cell harvesting step.

Over the years, MSCs have been tested in various Phase I and II clinical trials. However, the usage of MSCs for human treatment is still in a slow progress of meeting requirements for US Food & Drug Administration (FDA) approval as the efficacy of such treatment has yet to reach a satisfying level. The lack of efficacy could be attributed in part to the heterogeneous nature of MSCs, where it has been found that different subpopulations of MSCs even from a single human donor show varying phenotypes that affect treatment outcome (Poon, Z. et al., Stem Cells Transl. Med. 2015, 4, 56-65; and Rennerfeldt, D. A. et al., Stem Cells 2016, 34, 1135-1141). Sorting of cells based on phenotypes could be performed to enrich MSC population that has better performance in treatment (Poon, Z. et al., Stem Cells Transl. Med. 2015, 4, 56-65; and Lu, Y. et al., Lab Chip 2018, 18, 8778-889), but this approach generally has low cell manufacturing efficiency as a good amount of cells that have low treatment efficacy will be deemed as waste. Hence, it is necessary to find a way to maintain MSCs homogeneity throughout the course of culture.

Therefore, there is a need to develop new MCs for expansion and culture of MSCs to overcome the challenges listed above.

### SUMMARY OF INVENTION

It has been surprisingly found that the use of monodisperse microparticles that can be dismantled to readily release the desired MSCs can be developed. Said monodisperse microparticles solve some or all of the problems identified hereinbefore. Aspects and embodiments of the invention will now be discussed by reference to the following numbered embodiments.

1. Monodisperse microparticles suitable for use as microcarrier particles, the monodisperse microparticles comprising gelatin crosslinked by genipin, wherein the microparticles have:
a diameter of from 120 to 300 μm;
a Young's Modulus of from 10 to 110 kPa; and
a density of from 1.02 to 1.12 g/cm³, wherein
the microparticles have a coefficient of variation of less than or equal to 5% for the
diameter, and optionally one or both of the Young's modulus and density have a
coefficient of variation of less than or equal to 5%.

2. The microparticles according to Clause 1, wherein the diameter of the microparticles is from 140 to 300 μm.

3. The microparticles according to Clause 2, wherein the diameter of the microparticles is from 150 to 250 μm.

4. The microparticles according to Clause 3, wherein the diameter of the microparticles is from 175 to 225 μm.

5. The microparticles according to any one of the preceding clauses, wherein the Young's Modulus of the microparticles is from 10 to 50 kPa, such as from 50 to 90 kPa, such as from 95 to 105 kPa, such as from 90 to 100 kPa, such as around 100 kPa.

6. The microparticles according to any one of the preceding clauses, wherein the density of the microparticles is from 1.05 to 1.12 g/cm³, such as from 1.11 to 1.12 g/cm³.

7. The microparticles according to any one of the preceding clauses, wherein:
(a) the coefficient of variation of the microparticle diameter is from 0.5 to 4.8%, such as from 1 to 4.6%; or
(b) the coefficient of variation of the microparticle diameter and the Young's modulus of the microparticles is from 0.5 to 4.8%, such as from 1 to 4.6%; or
(c) the coefficient of variation of the microparticle diameter and density of the microparticles is from 0.5 to 4.8%, such as from 1 to 4.6%; or
(d) the coefficient of variation of the microparticle diameter, Young's modulus of the microparticles and density of the microparticles is from 0.5 to 4.8%, such as from 1 to 4.6%.

8. A method of forming monodisperse microparticles according to any one of Clauses 1 to 7, wherein the method comprises:
(a) providing uncrosslinked monodisperse gelatin microparticles; and
(b) crosslinking the uncrosslinked monodisperse gelatin microparticles in the presence of genipin and a solvent for a period of from 2 to 14 days, such as from 5 to 10 days, such as 10 days.

9. The method according to Clause 8, wherein the genipin is provided in a concentration of from 100 to 500 μg/mL in a solvent, such as from 200 to 400 μg/mL in a solvent, such as 250 μg/mL in a solvent.

10. A method of removing one or more adhered cells from a surface of a monodisperse microparticle according to any one of Clauses 1 to 7, the process comprising:
(a) providing a composition comprising a solvent, monodisperse microparticles according to any one of Clauses 1 to 7 and one or more cells attached to a surface of each monodisperse microparticle; and
(b) contacting the composition with a protease mixture (e.g. Pronase™) for a period of time to enzymatically degrade the monodisperse microparticles, thereby releasing one or more cells.

11. The method according to Clause 10, wherein the mixture of proteases (e.g. Pronase™) is added in a concentration of from 0.01 to 1 wt %, such as 0.1 wt % of the composition.

12. The method according to Clause 10 or Clause 11, wherein the monodisperse microparticles are present in a concentration of from 5 to 50 wt % of the composition.

13. The method according to any one of Clauses 10 to 12, wherein the period of time is from 1 to 60 minutes, such as from 2 to 30 minutes, such as 3 to 10 minutes, such as minutes.

14. The method according to any one of Clauses 10 to 13, wherein the method further comprises collecting the released cells from the composition, where the cells are substantially (e.g. entirely) free of residue from the monodisperse microparticles.

15. The method according to any one of Clauses 10 to 14, wherein the composition comprising a solvent, monodisperse microparticles according to any one of Clauses 1 to 7 and one or more cells attached to a surface of each monodisperse microparticle is obtained by adding cells and monodisperse microparticles according to any one of Clauses 1 to 7 to a culture medium for a period of time.

16. The method according to Clause 15, wherein the cells are added to the culture medium in an initial cell seeding density of from 1,000 to 10,000 cells/cm², such as 5,000 cells/cm².

17. The method according to Clause 15 or Clause 16, wherein the period of time for cell culturing is from 4 to 30 days, such as from 4 to 10 days.

### DRAWINGS

FIG. 1 shows Gelatin microcarriers (MCs) fabrication and comparison of diameter uniformity with existing MCs. (a) Gelatin microcarrier (MC) particles fabrication through droplet microfluidics. (b) Particle diameter uniformity of various MC particles: gelatin MC, SoloHill Collagen and Cytodex-3. (c) Particle diameter distribution of Cytodex-1, Cytodex-3, SoloHill and Gelatin MC. d) Coefficient of variation (CoV) indicates that Gelatin MC has the highest degree of diameter uniformity.

FIG. 2 shows the characterization of MSC culture on MCs. (a) Attachment efficiency of MSCs onto different types of MC particles after 24 hours; (b) Doubling time of MSCs when cultured on different types of MC particles; (c) Total MSC fold expansion on different MC particle types after 10 days of culture; (d) Growth curve of MSCs cultured on different of MC particle types from day 1 to day 10; (e) MSC proliferation as detected by WST-1 assay measured on day 1, 4, 7 and 10. Significant differences detected by day 10; and (f) Bright field images of MSC culture on different MC particle types on day 10. (*p<0.05, **p<0.01)

FIG. 3 shows the characterization of cell harvest from different MCs. (a) Harvest efficiency of MSC with Pronase™ enzymatic solution (30 minutes incubation); (b) Bright field time-lapse images of the dissolution process when incubating gelatin MCs in Pronase™ enzymatic solution; and (c) Overall yield of MSC expansion was reported to determine the ratio between total number of cells collected from MC culture on day 11 against initial cell seeding density. (*p<0.05, **p<0.01)

FIG. 4 shows the characterization of the differentiation potential of MSCs harvested from various 3D MCs and 2D planar culture. (a) Expression level of differentiation genes of MSC cultured in different conditions; (b) Images; and (c) Quantification of adipo-, osteo- and chondro-differentiation level were obtained through Oil Red O staining, Alizarin Red staining and sGAG assay. For Oil Red O staining, oil droplets were stained in red and cell nuclei were stained by hematoxylin in deep blue-purple; for Alizarin Red staining, calcium deposits were stained in red. (*p<0.05, **p<0.01)

FIG. 5 depicts flow cytometry for analyzing MSC heterogeneity. Flow cytometry analysis of MSC cultured on different types of MC particles and on 2D tissue culture flask (TCF) were performed to investigate heterogeneity of cell population by screening for (a) β-galactosidase; and (b) PPARY. Unstained MSC negative control in black, and positively stained MSC in red. CoV of marker expression among MSC population for (c) (β-galactosidase; and (d) PPARY were measured to determine heterogeneity of cell population after culturing on different MC particle types and on 2D TCF.

### Description

Monodisperse microparticles with specific features that enable a homogeneous batch of MSCs to be grown and which can then be readily “dissolved” to release the desired MSCs, thereby making the harvesting of the viable MSCs relatively easy are disclosed herein. Thus, disclosed herein are monodisperse microparticles suitable for use as microcarrier particles, the monodisperse microparticles comprising gelatin crosslinked by genipin, wherein the microparticles have:
a diameter of from 120 to 300 μm;
a Young's Modulus of from 90 to 110 kPa; and
a density of from 1.02 to 1.12 g/cm^{3 ,} wherein
the microparticles have a coefficient of variation of less than or equal to 5% for the diameter, and optionally one or both of the Young's modulus and density have a coefficient of variation of less than or equal to 5%.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

When used herein, the term “monodisperse microparticles” may refer to microparticles that have one or more properties that are essentially identical. For example, the of microparticles may have a property that has a coefficient of variation of 5% or less, such as from 0.5 to 4.8%, such as from 1 to 4.6%. For the avoidance of doubt, the level of variation may be determined inter-batch (i.e. microparticles obtained from different processing runs to generate substiantially identical materials) or, more particularly, intra-batch, where the microparticles are formed in the same processing run. When used herein, the term “coefficient of variation” refers to a statistical measure of the dispersion of data points in a data series around the mean. The coefficient of variation represents the ratio of the standard deviation to the mean and may be determined by formula (1):

$$C = \frac{σ}{μ} \qquad ( 1 )$$

where σ is the standard deviation and μ is the mean.

The microparticles disclosed herein may have any suitable diameter within the range of from 120 to 300 μm. For example, the microparticles may have a diameter of from 140 to 300 μm, such as from 150 to 250 μm, such as from 175 to 225 μm.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, disclosed herein are microparticles having a diameter of:
from 120 to 140 μm, from 120 to 150 μm, from 120 to 175 μm, from 120 to 250 μm, from 120 to 300 μm;
from 140 to 150 μm, from 140 to 175 μm, from 140 to 250 μm, from 140 to 300 μm;
from 150 to 175 μm, from 150 to 250 μm, from 150 to 300 μm;
from 175 to 250 μm, from 175 to 300 μm; and
from 250 to 300 μm.

The microparticles disclosed herein may have any suitable Young's Modulus within the range of from 10 to 110 kPa. For example, the microparticles may have a Young's Modulus of from 10 to 50 kPa, such as from 50 to 90 kPa, such as from 95 to 105 kPa, such as from 90 to 100 kPa, such as about 100 kPa.

The microparticles disclosed herein may have any suitable density within the range of from 1.02 to 1.12 g/cm³. For example, the microparticles may have a density of from 1.05 to 1.12 g/cm³, such as from 1.11 to 1.12 g/cm³.

As mentioned above, the microparticles disclosed herein may have at least one property with a low modulus of variation. However, in addition, one or more (e.g. two, three or all) of the properties may all display a low modulus of variation. For example:
(a) the coefficient of variation of the microparticle diameter may be from 0.5 to 4.8%, such as from 1 to 4.6%; or
(b) the coefficient of variation of the microparticle diameter and the Young's modulus of the microparticles may be from 0.5 to 4.8%, such as from 1 to 4.6%; or
(c) the coefficient of variation of the microparticle diameter and density of the microparticles may be from 0.5 to 4.8%, such as from 1 to 4.6%; or
(d) the coefficient of variation of the microparticle diameter, Young's modulus of the microparticles and density of the microparticles may be from 0.5 to 4.8%, such as from 1 to 4.6%.

For the avoidance of doubt, there is no need for the coefficients of variation for each of the properties mentioned above to be identical to one another. That is, the coefficients of variation may each be independently found within the ranges noted above. However, it is possible for the coefficients of variation for the selected properties to be substantially identical.

As will be appreciated, the monodisperse microparticles described herein are obtained using a suitable method for their formation. As such, there is further disclosed a method of forming monodisperse microparticles as described herein, wherein the method comprises:
(a) providing uncrosslinked monodisperse gelatin microparticles; and
(b) crosslinking the uncrosslinked monodisperse gelatin microparticles in the presence of genipin and a solvent for a period of from 2 to 14 days, such as from 5 to 10 days, such as 10 days.

Uncrosslinked monodisperse gelatin microparticles may be generated by any suitable method for use in the current invention. For example, using microfluidics. Particular examples of how this may be achieved in practice are provided in the examples section below.

Any suitable amount of genipin may be used to crosslink the uncrosslinked monodisperse gelatin microparticles. For example, the genipin may be provided in a concentration of from 100 to 500 μg/mL in a solvent, such as from 200 to 400 μg/mL in a solvent, such as 250 μg/mL in a solvent.

Further details of the formation of the microparticles may be obtained from the experimental section hereinbelow.

The monodisperse microparticles described herein may be particularly suited to adhering cells and allowing cells to grow on their surfaces. Details of how this cell adhesion and cell growth may be accomplished are provided in the experimental section hereinbelow. As will be appreciated however, it is important to be able to release the cells at an appropriate time. As such, there is a need for a method to be able to release the cells and so there is also disclosed herein a method of removing one or more adhered cells from a surface of a monodisperse microparticle as described herein, the process comprising:
(a) providing a composition comprising a solvent, monodisperse microparticles as described herein and one or more cells attached to a surface of each monodisperse microparticle; and
(b) contacting the composition with a protease mixture (e.g. Pronase™) for a period of time to enzymatically degrade the monodisperse microparticles, thereby releasing one or more cells.

The protease mixture may be formed from any suitable mixture of proteases suitable for the purpose. For example, the protease mixture may be the protease mixture Pronase™ Any suitable amount of protease mixture may be added to the monodisperse microparticles, provided that it is able to release the cells from the microparticles. For example, the mixture of proteases (e.g. Pronase™) may be added in a concentration of from 0.01 to 1 wt %, such as 0.1 wt % of the composition (e.g. the composition mentioned in step (a) above).

The monodisperse microparticles may form any suitable amount of the composition. For example, the monodisperse microparticles may be present in a concentration of from 5 to 50 wt % of the composition.

Any suitable period of time may be used in step (b) of the method above. For example, the period of time may be from 1 to 60 minutes, such as from 2 to 30 minutes, such as 3 to 10 minutes, such as 5 minutes.

As will be appreciated, after the cells have been released, it may be convenient and useful to collect said cells for further use and study. Thus, the method above may also further comprise collecting the released cells from the composition, where the cells are substantially (e.g. entirely) free of residue from the monodisperse microparticles.

As will be appreciated, the composition comprising a solvent, monodisperse microparticles as described herein and one or more cells attached to a surface of each monodisperse microparticle is obtained by adding cells and monodisperse microparticles as described herein to a culture medium for a period of time. For example, the cells may be added to the culture medium in an initial cell seeding density of from 1,000 to 10,000 cells/cm², such as 5,000 cells/cm². Additionally or alternatively, the period of time for cell culturing may be from 4 to 30 days, such as from 4 to 10 days. Further details of how this cell attachment and culturing on the surface of the monodisperse microparticles is achieved is provided in the experimental section hereinbelow.

Further aspects and embodiments of the invention will now be described by reference to the following non-limiting examples.

### EXAMPLES

### Materials

Gelatin powder, dimethyl sulfoxide, 1H, 1H, 2H, 2H-perfluoro-1-octanol, Sigmacote and Hoechst 33342 were purchased from Sigma-Aldrich, USA. Fluorocarbon oil HFE 7500 was purchased from 3M Novec™, Singapore. Pico-Surf™ 1 was purchased from Sphere Fluidics. Genipin was purchased from Challenge Bioproducts, Taiwan. Polyethylene (PE) tubing with an inner diameter of 0.38 mm was purchased from Scientific Commodities, USA. Dulbecco's Modified Eagle Medium (DMEM), fetal bovine serum (FBS), trypsin, Penincilin and Streptomycin were purchased from Gibco. Cytodex-1 and Cytodex-3 were purchased from GE Healthcare Life Sciences, USA. SoloHill (collagen coated) was purchased from Pall Corporation, USA. Trypan blue stain 0.4% was purchased from Life technologies, USA. WST-1 (4-[3-(4-lodophenyl)-2-(4-nitrophenyl)-2H-5-tetrazolio]-1,3-benzene disulfonate) was purchased from Roche. Adipo- and osteo-differentiation media were purchased from PromoCell. Lipid O Red O was obtained from BioVision Inc. Alizarin Red was purchased from ScienCell Research Laboratories. Glycosaminoglycan (sGAG) assay was purchased from Biocolor. Pronase™ was purchased from Sigma-Aldrich, USA, Calbiochem and Boehringer Mannheim. Alexa Fluor 647 Phalloidin, CellEvent Senescence Green Flow Cytometry Assay Kit, and Express One-Step SYBR GreenER Kit were purchased from Invitrogen, USA.

### Analytical Techniques

The particle diameter distribution of the prepared microcarriers (MCs) were analyzed with ImageJ (National Institutes of Health, USA) by randomly measuring the diameter of 300 microspheres from bright field images (4× magnification) of each MC type. Coefficient of variations (CoV) of the particle diameter for each MC types were measured by dividing the standard deviation of the diameter population by the mean diameter, as a mean to measure the degree of uniformity of the MC particle diameter. A smaller CoV indicates a more uniform particle diameter.

### Statistical Analysis

All biological data were expressed as arithmetic mean ±standard deviation. Where appropriate, ANOVA was used to analyse results with technical triplicates; when ANOVA analysis showed significant differences between independent experimental groups, post hoc Tukey tests were carried out to determine which experimental groups were significantly different with P<0.05 considered to indicate a statistically significant difference.

### Example 1. Fabrication of Gelatin MCs

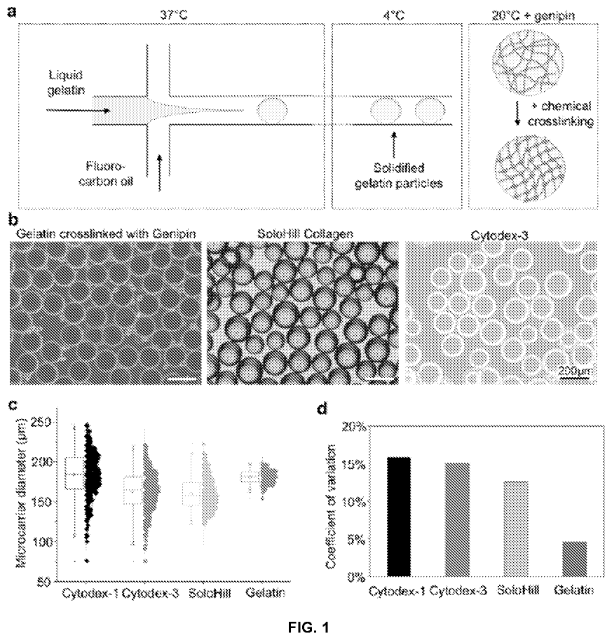

A microfluidic chip for gelatin droplet formation was fabricated as a polydimethylsiloxane (PDMS) cross-junction chip. The size of the channel can be designed to accommodate fabrication of gelatin particles of various sizes. Gelatin powder was dissolved in deionized (DI) water and heated to 50° C. to allow full dissolution of the powder. The dissolved gelatin solution was then transferred into a 5 mL glass syringe (Hamilton). Fluorocarbon oil HFE 7500 with Pico-Surf™ 1 (5% (w/w) in Novec™ 7500) was prepared in another 25 mL glass syringe. Both gelatin solution and fluorocarbon oil syringes were mounted onto two separate syringe pumps (Harvard, PHD2000), where the outlet of the syringes was connected to the microfluidic chip inlets via polyethylene (PE) tubing with an inner diameter of 0.38 mm. Flow rates for infusing gelatin solution and fluorocarbon oil were set at 2.5 μL/min and 12.5 μL/min, respectively. Inside the microfluidic chip, gelatin solution was pinched by fluorocarbon oil introduced from the sides of the cross-junction channel to generate gelatin particles with a diameter of 180 μm (FIG. 1a). The droplet generation process was carried out in a 37° C. warm room to maintain the liquid state of the gelatin solution. The generated droplets were collected into a 50 mL tube (Falcon) placed in an ice box to allow solidification of the particles. Upon gelation, the solidified gelatin particles were washed with 1H, 1H, 2H, 2H-perfluoro-1-octanol and DI water to remove excessive fluorocarbon oil and to suspend the particles into aqueous solution. The gelatin particles were then crosslinked with 250 μg/mL genipin (which was dissolved in dimethyl sulfoxide at 50 mg/mL) for at least two days with constant shaking to prevent aggregation. The genipin-crosslinked gelatin particles were washed with DI water and sterilised with 70% (v/v) ethanol prior to cell culture.

### Characterization

The CoV for the particle diameter distribution was 4.52% for Gelatin MCs (FIG. 1b-c). In principle, the diameter of the Gelatin MC is tunable as it depends on the channel size of the microfluidic chip designed for particle. With the use of droplet microfluidics platform, the fabricated particle diameter showed high uniformity, with CoV below 5%.

### Example 2. Preparation of Commercialized MCs

Cytodex-1, Cytodex-3 and SoloHill (collagen coated) were supplied in dry particles form. Each MC type was weighed accordingly and with the information of surface area per mass given by the manufacturers, a total surface area of 500 cm² for MC cell culture was obtained. Cytodex-1 and Cytodex-3 were hydrated in 15 mL of PBS for two hours to obtain the fully swollen state of the particles. Solohill MC was prepared in 15 mL of PBS as well. All three MCs were autoclaved for 30 minutes at 121° C. for sterilization. Subsequently, MCs were washed once with PBS and thrice with culture medium (DMEM with 10% FBS). MCs in culture medium were then incubated in a 37° C. and 5% CO₂ incubator (Thermo Scientific) for two hours for the conditioning of MCs prior to cell seeding.

### Characterization

The CoV for the particle diameter distributions were 15.87%, 15.07% and 12.55% for Cytodex-1, Cytodex-3 and SoloHill MCs, respectively (FIG. 1b-c). According to the specifications given on each of the commercialized MC, Cytodex-1 MCs have a particle diameter range of 131-220 μm; Cytodex-3 from 133 to 215 μm; and SoloHill from 125 to 212 μm.

### Example 3. Mesenchymal Stromal Cell (MSC) Expansion on MCs

### MSC 2D Planar Culture

Bone marrow (BM)-derived MSCs were obtained from a commercial source (Lonza) as passage 1. For the first culture passage, MSCs were isolated from a healthy donor derived BM aspirate through ficoll density centrifugation and plastic adherence. Subsequently, MSCs were expanded to passage 4 on polystyrene TCF with cell seeding density of 1000 cells/cm². MSCs were cultured in a 37° C. and 5% CO₂ incubator (Thermo Scientific) for each passage until 90% confluence with culture medium supplied at 0.2 mL/cm². Low glucose DMEM supplemented with 10% FBS and 5% Penincilin/Streptomycin was used for MSCs culture and expansion. For cell passaging, trypsin was added to the culture plate for 5 minutes and incubated at 37° C. with 5% CO₂. For cell count, 10 μL was taken out from the cell solution and mixed with 10 μL of Trypan blue stain 0.4%. The mixed solution was then loaded onto a cell counting chamber slide and read by an automated cell counter (Cellometer Auto-T4, Nexcellom Bioscience). Automated cell counting readings were cross-checked with manual cell counting on a hemocytometer C-chip under an inverted bright-field microscope.

### MSC MC Culture

Spinner flasks (Corning) was pre-coated with Sigmacote to siliconize the glass surface to prevent adhesion of cells or MSCs onto the glass wall. The spinner flasks were then rinsed with DI water and autoclaved prior to use for cell culture. Before adding cells into the flasks, the prepared MCs were added to the spinner flask with a total volume of 25 mL. The spinner flasks were placed on top of a magnetic stirrer (Multimagstir Genie, Scientific Industries) and set to a rotation speed of 30 rpm to fully suspense the MCs. For cell preparation, MSCs were harvested from 2D planar culture at the end of passage 4. Cell counting were performed to obtain a cell concentration of 0.1 million cells/mL in 25 mL of culture medium. Cell suspension was then transferred to each flask to reach a working volume of 50 mL with a cell seeding density of 5000 cells/cm². An intermittent agitation approach was applied to facilitate attachment of MSCs onto MCs for the first 24 hours, where 2 minutes of 30 rpm stirring was followed by 28 minutes of static rest. At the end of day 1 (after 24 hours of intermittent agitation), MCs were allowed to settle down inside the spinner flask for 5 minutes and 30 mL of supernatants were carefully aspirated without collecting the MCs to measure the number of cells which did not attach onto the MCs for cell attachment efficiency measurement. After that, culture medium was added into the flasks to a total volume of 100 mL and a continuous stirring (30 rpm) was performed for the whole dynamic culture (11 days). Medium exchanged was performed on day 4, 7 and 10, where 50% of the volume was replaced with fresh culture medium.

### Sampling of MC Culture

During the dynamic culture period, 1 mL of MC suspensions were collected for each MC type for cell counting. A protease mixture known as Pronase™ was added at 0.1% concentration to release the cells from the MC particles with 30 minutes incubation. Pronase™ is a proteolytic enzyme obtained from Streptomyces griseus (Ishii, S. et al., Chapter 116—Griselysin. Handbook of Proteolytic Enzymes 3rd Edition, Volume 1, 572-574, (2013)) and it showed good cell releasing performance from MC particles. As only 1% of MC suspension was retrieved from the whole culture (1 mL out of 100 mL), both MCs and MSCs were centrifuged and resuspended in 100 μL of culture medium to reach a cell concentration that was desirable for cell counting. Further to this, the MCs and MSCs suspension were directly aliquoted and injected into a counting chamber for cell counting without sorting out the cells from the MCs by passing them through a cell strainer. This was to reduce the amount of cell lost during the process of cell-particle separation, and this yielded a closer resemblance to the actual total cell number in the MC culture.

### WST-1 Assay for Cell Growth Monitoring

WST-1 assay is a colorimetric assay used to quantify cell proliferation and growth. It measures the overall activity of mitochondrial dehydrogenases where the tetrazolium salts WST-1 are cleaved to formazan by cellular enzymes (Berridge, M. V. et al., The Biochemical and Cellular Basis of Cell Proliferation Assays That Use Tetrazolium Salts. Biochemica, 4, 15-19, (1996)). On day 1, 4, 7 and 10, 1 mL of MC suspension was aliquoted from the spinner flask and transferred into a 1.5 mL tube (Axygen). The MC suspension was further divided and transferred into three 48-well plate wells at a volume of 300 μL for triplicate examination of WST-1 activity. The cell proliferation reagent WST-1 was added to each sample at 1:10 dilution, and the well plate was placed inside a 37° C. with 5% CO₂ incubator for three hours. After incubation, absorbance of the samples was measured at 440 nm with a microplate reader (Tecan).

### Confocal Imaging

On day 1, 4, 7 and 10, 1 mL of MC suspension was aliquoted from the spinner flask and transferred into a 1.5 mL tube (Axygen). MSCs on MCs were washed once with PBS before 4% (w/v) formaldehyde was added to fix the cells on the particles for 20 minutes at room temperature. This was followed by another 15 minutes of incubation with 0.1% (v/v) Triton-X in PBS to permeate cell membranes for actin staining. Next, MSCs on MCs were washed thrice with PBS and stored in PBS with 5% (v/v) penincilin-streptomycin. To prepare for confocal imaging, the fixed MSCs on MCs were incubated with 10 μg/mL of Alexa Fluor 647 Phalloidin and 1 μg/mL of Hoechst 33342 in PBS for 24 hours at room temperature (25 ° C.). After staining, the MSCs on MCs were washed with PBS thrice to remove excessive unbound dyes. MSCs were then imaged with a confocal microscope LSM800 (Zeiss, Germany), and the collected images were processed with IMARIS 9.3 (Oxford Instruments, United Kingdom).

### Results and Discussion

The material comprising MC plays a role in affecting cell adhesion as well as cell growth. Gelatin has been used widely as scaffold or coating material for cell culture, thus it is not surprising that gelatin offers good cell attachment efficiency as well as proliferation rate. MSCs cultured on gelatin has been shown to enhance chondro- and osteo-differentiation while maintaining similar level of adipodifferentiation when compared to 2D monolayer culture. Gelatin has also been demonstrated as a substrate for 2D monolayer culture and adipogenesis and osteogenesis differentiation was enhanced (Obara, C. et al., Stem Cells Int. 2016, 2016, Article ID 9013089; and Khan, I. U. et al., Turk. J. Biol. 2017, 41, 969-978). This suggested to us that gelatin is a suitable substrate for the growth of MSC while maintaining its differentiation capability and multipotency. In addition, as gelatin is seen widely as a safe material for human exposure and consumption, there is relatively low but non-zero risk in delivery any degraded gelatin with the therapeutics of cell suspension. Therefore, gelatin was used to fabricate Gelatin-based MCs.

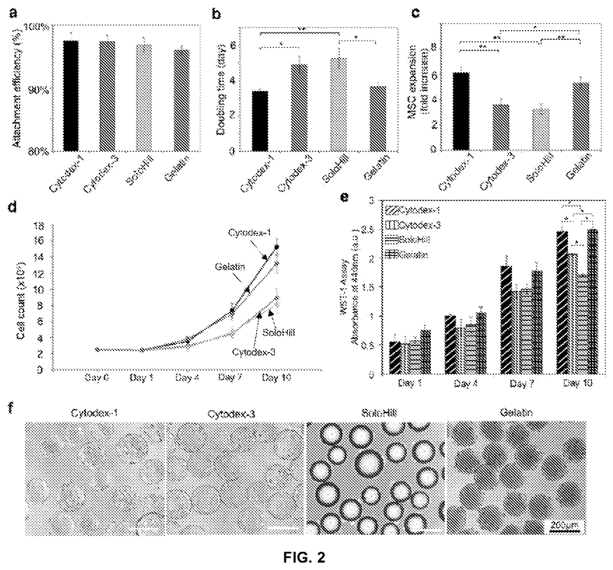

We first investigated the attachment efficiency of MSCs onto different types of MCs after 24 hours of intermittent agitation inoculation. The results showed that the attachment efficiencies of MSCs onto MC were generally >95% (Cytodex-1: 97.52±0.29%, Cytodex-3: 97.41±0.16%, SoloHill: 96.84±0.16%, Gelatin: 95.62±0.18%) (FIG. 2a). Subsequently, we conducted a dynamic culture of MSCs with MC in spinner flasks to investigate the cell proliferation performance on these different MCs. During the course of culture, we harvested 1 mL of MC suspension from each condition on day 1, 4, 7 and 10 to perform cell counting. FIG. 2d shows MSCs proliferation on four different types of MCs, and the results showed that Cytodex-1 and Gelatin MCs had similar performance in terms of promoting cell growth. Cell growth on Cytodex-3 and SoloHill MCs were slower as compared to Cytodex-1 and Gelatin MCs.

We also conducted an offline analysis by collecting 1 mL of MC suspension for WST-1 assay, where metabolic activity of MSCs cultured on different MCs were measured. The observation of WST-1 activity of MSCs was consistent with our cell counting, where MSCs cultured on Cytodex-1 and Gelatin MCs showed higher overall activity which translated to greater total cell numbers (FIG. 2e). Bright field images (FIG. 2f) and confocal images of the MC cultures also provided a mean to estimate cell number per particle, and the imaging results were in concordance with the previous cell counting and WST-1 measurements.

Population doubling time on each MC type was calculated when cell growth was in the exponential phase (day 1 to day 10). Population doubling times for Cytodex-1, Cytodex-3, SoloHill and Gelatin MCs were 3.41±0.13 days, 4.89±0.50 days, 5.27±0.55 days and 3.67±0.19 days, respectively (FIG. 2b). On day 11, MSCs were harvested from the culture and a cell-particle filtering process was conducted to separate MSCs from the MC. This contributed to a decrease in total cell number collected at this step. Cell expansion fold was calculated by obtaining the ratio of cell number on day 10 to the initial cell number seeded. The expansion-folds were 6.11±0.42 for Cytodex-1, 3.57±0.47 for Cytodex-3, 3.26±0.43 for SoloHill, and 5.28±0.48 for Gelatin MC (FIG. 2c).

### Example 4. MSC Harvest and Cell Recovery Efficiency

### MSC Harvest from MC Culture

MSCs cultured on MCs in Example 3 were allowed to settle down inside the spinner flask before transfer into a 50 mL tube. The MCs were allowed to sediment to the bottom of the tube before supernatants were removed, and PBS was added to wash the particles and the cells. MSCs release with Pronase™ was performed for 30 minutes inside a 37° C. incubator with occasional agitation. Subsequently, the whole suspension was filtered through a 70 μm cell strainer (SPL Life Sciences) to separate the cells from the MCs. In the case of Gelatin MC, all MCs were dissolved in Pronase™ solution and cell suspension was filtered through a 70 μm cell strainer to remove large cell clumps.

### Results and Discussion

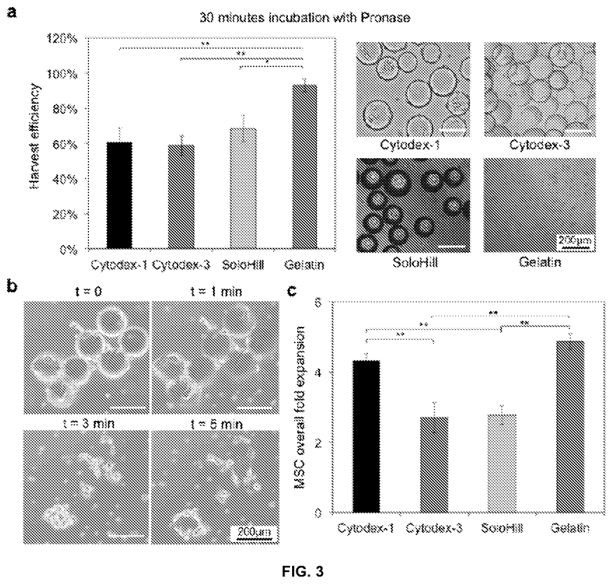

Downstream processing of cell manufacturing involves cell harvesting after cell expansion. Various groups have reported the use of different enzymatic solutions such as trypsin (Rafiq, Q. A. et al., Biotechnol. J. 2016, 11, 473-486; and Lawson, T. et al., Biochem. Eng. J. 2017, 120, 49-62), TrypLE (Carmelo, J. G. et al., Biotechnol. J. 2015, 10, 1235-1247; and Rafiq, Q. A. et al., Biotechnol. Bioeng. 2017, 114, 2253-2266), and collagenase (Goh, T. K.-P. et al., BioRes. Open Access 2013, 2, 84-97). We tested a few enzymatic solutions and our results suggested that Pronase™ showed the best performance in detaching MSCs from MC. Conventionally, a separation step is necessary to sort out the cells from the MC particles after the enzymatic harvesting process. Cell strainers with pore diameter in between the size range of 50-100 μm could be used to separate the cells from the particles. In this process of cell recovery with enzymatic incubation and filter membrane sorting, there is a huge cell loss due to two reasons: (1) cells do not fully detach from the MC particles even after treating with enzyme. This was especially prominent for the Cytodex particles in our result (FIG. 3a); and (2) the filtering step with a cell strainer could lead to significant cell loss during the cell-particle separation as cells could be stuck in between particles and the filter membrane. Typically, a 60-70% cell recovery is expected from MC culture by such separation protocols (Weber, C. et al., Open Biomed. Eng. J. 2007, 1, 38-46). In contrast, Gelatin MC were completely dissolvable with Pronase™ treatment. The particles could be fully dissolved in 5 minutes with 0.1% Pronase™ solution, thus eliminating the need to perform separation of cells from the particles (FIG. 3b).

To quantify the cell harvest efficiency process on each type of MCs, we seeded 0.4 million of MSCs and MCs with total surface area of 50 cm² into a 24-well ultra-low attachment well plate (Corning) for overnight incubation inside a 37° C. with 5% CO₂ incubator. Cell proliferation in the first 24 hours was minimal and this allowed us to approximate the total cell number that could be harvested from the MCs when we performed the enzymatic detachment for the cell harvest step and cell-particle separation in the filtering step. For all conditions, incubation time with Pronase™ was set to be 30 minutes and all detached cells were passed through a 70 μm cell strainer to filter out MC particles or cell aggregates. Our results showed that the overall cell recovery efficiencies were 60.55±8.25%, 58.75±5.74%, 68.41±7.48%, 92.95±3.56% for Cytodex-1, Cytodex-3, SoloHill and Gelatin MC, respectively. As a result, the overall efficient cell yield in terms of fold expansion were 4.32±0.21 for Cytodex-1, 2.70±0.43 for Cytodex-3, 2.77±0.26 for SoloHill, and 4.87±0.23 for Gelatin MC after the cell recovery step (FIG. 3c). The direct dissolution of the Gelatin MCs allowed efficient recovery of cells and thus led to a higher overall cell yield.

### Example 5. Multipotency and Differentiation Capability of MSC Cultured on Various Type of MCs

### MSC Differentiation Induction and Quantitative Measurement

After cell harvest with Pronase™ described in Example 3, MSCs were re-plated at cell seeding densities of 3×10⁴ cells/cm² and of 1.5×10⁴ cells/cm² in a 24 well plate (Thermo Fisher) for adipo- and osteo-differentiation induction, respectively. MSCs were initially cultured with DMEM and 10% FBS culture medium to allow attachment of cells onto the plate. After 24 hours, culture media were replaced with adipo- and osteo-differentiation media respectively to initiate differentiation. For chondro-differentiation, MSCs were transferred into a 96 ultra-low attachment round well plate (Corning) at 1.5×10⁵ cells/cm² cells. MSCs were spun-down at 1200 rpm for 10 minutes to obtain cell pellets. Chondrogenic differentiation induction medium was then added into the well at 150 μL to initiate chondrogenic differentiation. Adipo- and osteo-differentiation were both performed for 10 days and media were changed every two days. Chondrodifferentiation was conducted for 14 days and media were changed every day. Each condition was conducted in a triplicate fashion. After each differentiation, quantification of adipo-, chondro- and osteo-differentiation were conducted using Lipid O Red O Alizarin Red and sGAG, respectively, according to each manufacturer's protocol.

### Real-time Quantitative Polymerase Chain Reaction (RT-qPCR)

Total RNA was extracted from MSCs using QIAGEN RNeasy Mini Kit and RNA to cDNA reverse transcription was performed using BioRAD iScript gDNA Clear cDNA Synthesis Kit with a BioRad C1000 Thermal Cycler. The relative amount of each transcript was determined by real-time RT-PCR using the ABI StepOnePlus instrument, and the Express One-Step SYBR GreenER Kit, according to the manufacturer's protocol. The 2-ddCt method was used to analyze the results using GAPDH as the housekeeping gene. RT-qPCR was performed in triplicate and thermal cycle conditions were 50° C. for 2 minutes, 95° C. for 10 minutes, then 50 cycles at 95° C. for 15 seconds and 60° C. for 1 minute.

### Results and Discussion

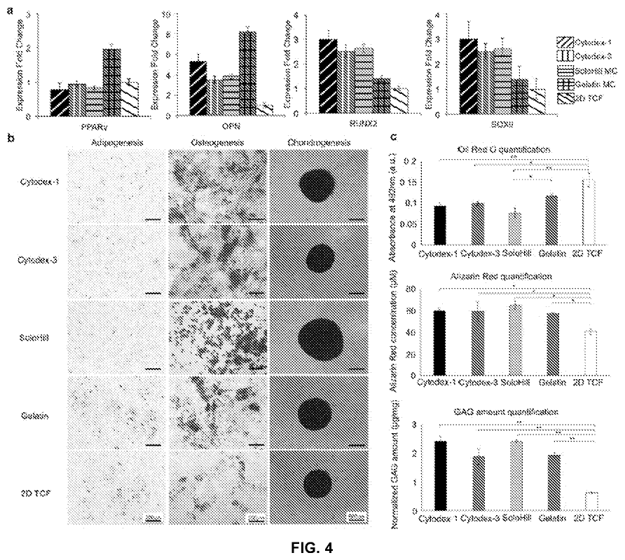

MSCs exhibit in vitro differentiation upon chemical induction along the tissue cell lineages of bone, cartilage, and fat, and this attribute is termed trilineage multipotency. Thus, we evaluated the multipotency of the MSCs cultured on different types of MC and 2D monolayer culture by inducing differentiation into the three lineages. We employed PCR to determine the expression level of genes associated to differentiation towards the adipo-(PPAR_{Y}), chondro-(SOX9) and osteo-lineages (OPN and RUNX2). The results showed that cells cultured on MC generally showed higher chondro- and osteo-differentiation genes level (SOX9, OPN and RUNX2). For adipo-differentiation, MSCs on commercial MC showed lower expression of PPAR_{Y} while Gelatin MC-cultured MSCs showed an upregulation in PPAR_{Y} expression when compared to 2D TCF culture (FIG. 4a).

We conducted further experiments to induce trilineage differentiation of MSCs harvested from the three mentioned conditions. Adipo- and osteo-differentiation were carried out for 10 days while chondro-differentiation was performed for 14 days. Alizarin Red and Lipid O Red O staining were performed on day 10 for osteo- and adipo-differentiated cells, while sGAG contents were measured from the chondro-pellets at day 14. The sGAG contents were normalized to the pellet weight. MC-cultured cells exhibited higher calcium deposits and normalized sGAG content when compared to 2D TCF-cultured cells, as measured by Alizarin Red staining and sGAG assay, respectively. However, Cytodex-1, Cytodex-3 and SoloHill cultured-cells showed lower oil deposits after 10 days of adipo-differentiation when compared to 2D TCF-cultured cells (FIG. 4b). Gelatin MC-cultured MSCs produced less oil droplets than 2D TCF-cultured cells, but the difference was not statistically significant. While most commercial MCs showed similar trend in promoting chondro- and osteo-differentiations and losing certain degree of adipogenesis differentiation capability, Gelatin MC-cultured MSCs seem to retain a higher degree of trilineage multipotency with a more balanced differentiation performance when compared to both commercial MC and 2D TCF cultures.

### Example 6. Effect of MC Culture on MSC Population Heterogeneity

### Flow Cytometry

After cell harvest with Pronase™ as described in Example 3, MSCs were fixed with 4% paraformaldehyde for 20 minutes, followed by permeabilization of cells with ice-cold 100% methanol. The concentration and incubation of antibody PPARY (Thermo Fisher) were conducted according to manufacturer's protocol. Secondary antibody (Alexa Fluor 647) was added to cell sample for PPARY staining as required. Senescence marker (β-galactosidase staining was carried out using CellEvent Senescence Green Flow Cytometry Assay Kit, according to the manufacturer's protocol. After preparation of samples, the unstained cell population was run as a negative control with Accuri® C6 Flow Cytometer for gating and threshold determination for positive and negative populations. A total of 20,000 events were measured for each antibody staining.

### Results and Discussion

Heterogeneity in culture-expanded MSC populations is a well-known issue and has been reported in several studies and review papers (Whitfield, M. J. et al., Stem Cell Res. 2013, 11, 1365-1377; and Rennerfeldt, D. A. et al., Stem Cells 2016, 34, 1135-1141). Studies suggested that heterogeneity among MSCs could affect disease treatment efficacy, and a functional MSC population with more narrowly defined phenotypic distribution with certain characteristics could improve treatment outcome (Poon, Z. et al., Stem Cells Transl. Med. 2015, 4, 56-65; and Lu, Y. et al., Lab Chip 2018, 18, 8778-889). Therefore, we evaluated the effect of MC culture on MSC population heterogeneity by using flow cytometry analysis. CoV, which is the ratio of the standard deviation to the mean of the fluorescent readouts from the stained markers on single cell, indicates the dispersion of the data point. We calculated the CoV to measure the degree of dispersion of the data point to represent the heterogeneity of the population. We fixed MSCs harvested from either MC culture or 2D monolayer culture, and conducted intracellular staining for PPAR_{Y}.

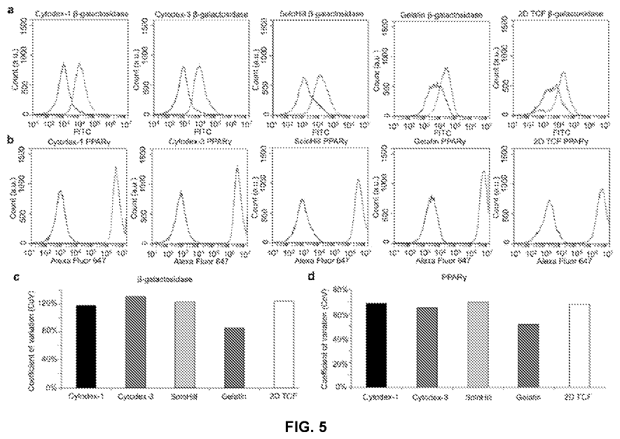

We also conducted a (β-galactosidase staining to evaluate the senescence level of the MSC population. Each staining was conducted individually and unstained MSCs were used as negative control (black line in FIG. 5a and b). CoV for (β-galactosidase fluorescence readouts were 117.61% for Cytodex-1, 130.54% for Cytodex-3, 123.11% for SoloHill, 85.70% for Gelatin MC, and 123.94% for 2D TCF culture; CoV for PPAR_{Y} fluorescence readouts were 68.46% for Cytodex-1, 64.83% for Cytodex-3, 69.73% for SoloHill, 51.24% for Gelatin MC, and 67.70% for 2D TCF culture. Thus, Gelatin MC-cultured MSCs had the lowest CoV for β-galactosidase (FIG. 5c) and PPARY (FIG. 5d). CoVs among other commercial MC and 2D monolayer culture were similar. This suggested that the difference between 3D MC culture and 2D monolayer culture was not the main factor which contributed to a difference in the degree of heterogeneity among MSC population. One possible explanation would be the high diameter uniformity of the fabricated Gelatin MC that provided a more uniform set of physical cues environment for MSCs. This could indicate the importance of having a tighter size range for MC fabrication. In addition, Gelatin MC-cultured MSC showed a lower expression level of β-galactosidase (FIG. 5a). This indicated that MSCs cultured on Gelatin MC could maintain multipotency, as multipotency is inversely correlated to senescence (Stolzing, A. et al., Rejuvenation Res. 2006, 9, 31-35; and Li, Y. et al., Int. J. Mol. Med. 2017, 39, 775-782). On the other hand, commercial MC-cultured MSCs seem to induce a high level of senescence among MSC population when compared to MSCs cultured on Gelatin MC or 2D TCF.

1. Monodisperse microparticles suitable for use as microcarrier particles, the monodisperse microparticles comprising gelatin crosslinked by genipin, wherein the microparticles have:
a diameter of from 120 to 300 μm;
a Young's Modulus of from 10 to 110 kPa; and
a density of from 1.02 to 1.12 g/cm³, wherein
the microparticles have a coefficient of variation of less than or equal to 5% for the diameter, and optionally one or both of the Young's modulus and density have a coefficient of variation of less than or equal to 5%.

2. The microparticles according to claim 1, wherein the diameter of the microparticles is from 140 to 300 μm.

3. The microparticles according to claim 2, wherein the diameter of the microparticles is from 150 to 250 μm.

4. The microparticles according to claim 3, wherein the diameter of the microparticles is from 175 to 225 μm.

5. The microparticles according to claim 1, wherein the Young's Modulus of the microparticles is from 10 to 50 kPa, such as from 50 to 90 kPa, such as from 95 to 105 kPa, such as from 90 to 100 kPa, such as around 100 kPa.

6. The microparticles according to claim 1, wherein the density of the microparticles is from 1.05 to 1.12 g/cm³, such as from 1.11 to 1.12 g/cm³.

7. The microparticles according to claim 1, wherein:
(a) the coefficient of variation of the microparticle diameter is from 0.5 to 4.8%, such as from 1 to 4.6%; or
(b) the coefficient of variation of the microparticle diameter and the Young's modulus of the microparticles is from 0.5 to 4.8%, such as from 1 to 4.6%; or
(c) the coefficient of variation of the microparticle diameter and density of the microparticles is from 0.5 to 4.8%, such as from 1 to 4.6%; or
(d) the coefficient of variation of the microparticle diameter, Young's modulus of the microparticles and density of the microparticles is from 0.5 to 4.8%, such as from 1 to 4.6%.

8. A method of forming monodisperse microparticles according to claim 1, wherein the method comprises:
(a) providing uncrosslinked monodisperse gelatine microparticles; and
(b) crosslinking the uncrosslinked monodisperse gelatine microparticles in the presence of genipin and a solvent for a period of from 2 to 14 days, such as from 5 to 10 days, such as 10 days.

9. The method according to claim 8, wherein the genipin is provided in a concentration of from 100 to 500 μg/mL in a solvent, such as from 200 to 400 μg/mL in a solvent, such as 250 μg/mL in a solvent.

10. A method of removing one or more adhered cells from a surface of a monodisperse microparticle according to claim 1, the process comprising:
(a) providing a composition comprising a solvent, monodisperse microparticles according to claim 1 and one or more cells attached to a surface of each monodisperse microparticle; and
(b) contacting the composition with a protease mixture for a period of time to enzymatically degrade the monodisperse microparticles, thereby releasing one or more cells.

11. The method according to claim 10, wherein the mixture of proteases is added in a concentration of from 0.01 to 1 wt %, such as 0.1 wt % of the composition.

12. The method according to claim 10, wherein the monodisperse microparticles are present in a concentration of from 5 to 50 wt % of the composition.

13. The method according to claim 10, wherein the period of time is from 1 to 60 minutes, such as from 2 to 30 minutes, such as 3 to 10 minutes, such as 5 minutes.

14. The method according to claim 10, wherein the method further comprises collecting the released cells from the composition, where the cells are substantially free of residue from the monodisperse microparticles.

15. The method according to claim 10, wherein the composition comprising a solvent, monodisperse microparticles and one or more cells attached to a surface of each monodisperse microparticle is obtained by adding cells and monodisperse microparticles to a culture medium for a period of time.

16. The method according to claim 15, wherein the cells are added to the culture medium in an initial cell seeding density of from 1,000 to 10,000 cells/cm², such as 5,000 cells/cm².

17. The method according to claim 15, wherein the period of time for cell culturing is from 4 to 30 days, such as from 4 to 10 days.
